(19) United States
(12) Patent Application Publication
(10) Pub. No.: US 2018/0280067 A1
(43) Pub. Date: Oct. 4, 2018

(54) HEXALOBE SCREW DRIVER

(71) Applicant: Spineology Inc., St. Paul, MN (US)

(72) Inventors: Todd BJORK, Hudson, WI (US); Steve JACOBSON, Lake Elmo, MN (US); Troy KNAPP, Glenwood City, WI (US)

(21) Appl. No.: 15/939,268

(22) Filed: Mar. 28, 2018

Related U.S. Application Data
(60) Provisional application No. 62/480,134, filed on Mar. 31, 2017.

(51) Int. Cl. A61B 17/88

(52) U.S. Cl. CPC A61B 17/888; A61B 17/8888

(57) ABSTRACT

A hexalobe screw driver for use with a screw head having a hexalobe socket. The hexalobe screw driver includes a split tip. An alignment nub disposed on the distal end of the driver tip. The alignment nub may interface with a nub socket in a hexalobe screw socket head.

### PRIORITY

This application claims the benefit of U.S. Provisional Application Ser. No. 62/480,134, filed on Mar. 31, 2017, which is hereby incorporated herein by reference in its entirety.

### FIELD

The present invention generally relates to a screw driver having a hexalobe tip. More particularly, the present invention relates to a hexalobe screw driver having a pilot nub and self-retaining tension that may be used for placing surgical implants.

### BACKGROUND

It is desirable to maintain correct alignment between a screw driver and a screw. This need is particularly important when placing implants in surgical procedures. Hexalobe screw sockets can be shallow when a low profile screw head is desired. Low profile screws heads are advantageous in surgical procedures because the low profile allows the implant to disrupt the least amount of patient anatomy. Because of the shallow socket depth, the tension force of driving the screw can cause misalignment and currently available hexalobe driver designs risk twisting out of the screw head when in use. Patient injury can result from a misaligned screw driver. Thus, there is a need for a screw driver that has a hexalobe tip that is retained within a low profile screw having a hexalobe head.

### SUMMARY

The disclosure includes a hexalobe screw driver for use with a screw head having a hexalobe socket. In an example embodiment of the present invention, the hexalobe screw driver includes a driver tip that includes a split. The disclosure further includes an alignment nub disposed on the distal end of the driver tip. The alignment nub may interface with a nub socket in a hexalobe screw socket head.

The disclosure includes a hexalobe screw driver, comprising a driver tip, the driver tip defining a hexalobe engagement feature at a distal end portion of the driver tip. A longitudinal split is defined through the tip from the distal end portion thereof and extending proximally into the driver tip. The hexalobe engagement feature includes an alignment nub protruding distally beyond the distal end portion of the engagement feature.

The disclosure additionally includes a hexalobe screw system, comprising a screw driver tip and hexalobe screw. The screw driver tip defines a hexalobe engagement feature at a distal end of the driver tip. A longitudinal split is defined through the tip from the distal end thereof and extends proximally into the driver tip. The hexalobe engagement feature includes an alignment nub protruding distally beyond the distal end of the engagement feature. The hexalobe screw includes a head portion forming a proximal portion of the screw and a threaded portion forming the distal portion of the screw. A socket is defined inward from a proximal end into the head portion. The socket is sized and shaped to receive and engage the hexalobe engagement feature of the screw driver tip.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a driver tip according to certain embodiments.

FIG. 2 depicts a perspective view of a screw for use with the driver tip according to certain embodiments.

FIG. 3 depicts a cross sectional view of a driver tip engaged with a screw according to certain embodiments.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims. For illustrative purposes, cross-hatching, dashing or shading in the figures is provided to demonstrate sealed portions and/or integrated regions or devices for the package.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

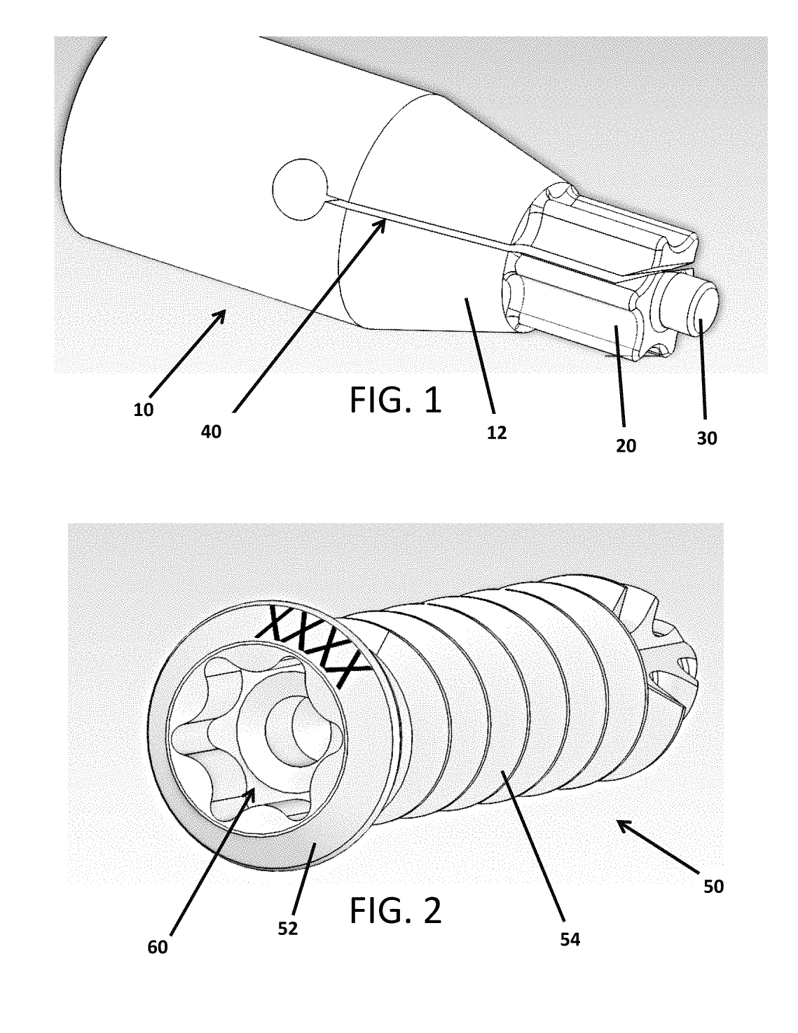

Referring to FIG. 1, a hexalobe screw driver 10 is shown. The driver 10 includes a driver tip 12 at a distal end thereof. Driver tip 12 includes a hexalobe engagement feature 20 defined at its distal end. Driver tip 12 includes a split 40 running longitudinally through the tip and proximally into the body of the screw driver 10. The hexalobe engagement feature 20 includes an alignment nub 30 protruding distally beyond the end of the engagement feature 20.

Referring now to FIG. 2, a hexalobe screw 50 is shown. Screw 50 includes a hexalobe screw head 52 portion defining the proximal portion of the screw and a threaded portion 54 forming the distal portion of the screw. A socket 60 is defined inward from the proximal end into the screw head 52. The socket 60 is sized and shaped to receive and engage the driver tip 12, including nub 30, of the screw driver 10 in FIG. 1.

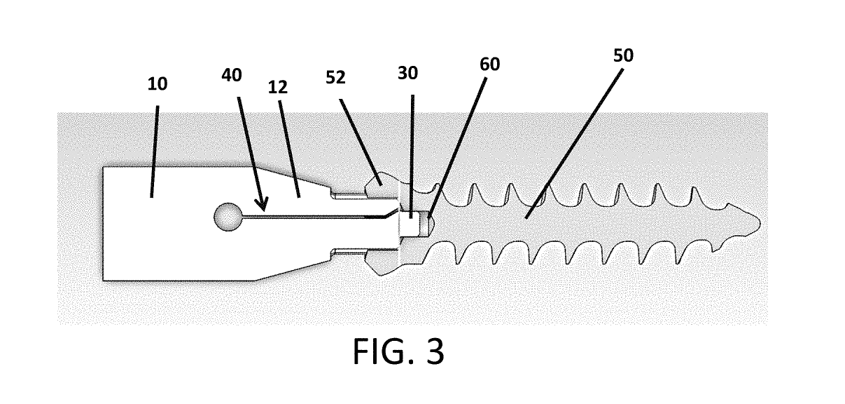

FIG. 3 depicts a cross-sectional view of driver tip 12 disposed within and engaged with the screw 50. Nub 30 can be seen seated in nub socket 60.

In use, the split 40 may be splayed open to create an interference fit between driver tip 12 and the socket 60 of screw 50, thus securely retaining the screw 50 onto tip 12. Once placed into nub socket 60, nub 30 creates tighter axial alignment, maintaining the desired alignment between screw driver 10 and screw 50. Because the desired alignment between driver 10 and screw 50 is retained, neither screw 50 nor driver 10 will rotate off axis under driving force. The result is that driver 10 and screw 50 stay aligned, do not separate during driving force and screw 50 is held much tighter to tip 12.

Nub socket 60 creates greater holding depth in screw head 52, without increasing the overall height of screw head 52. This allows for screw 50 to maintain its low profile, conserving patient anatomy and/or creating the least amount of disruption to whatever environment screw 50 is placed.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A hexalobe screw driver, comprising:
a driver tip, the driver tip defining a hexalobe engagement feature at a distal end portion of the driver tip,
wherein a longitudinal split is defined through the tip from the distal end portion thereof and extending proximally into the driver tip, and
wherein the hexalobe engagement feature includes an alignment nub protruding distally beyond the distal end portion of the engagement feature.

2. A hexalobe screw system, comprising:
a screw driver tip, defining a hexalobe engagement feature at a distal end of the driver tip, wherein a longitudinal split is defined through the tip from the distal end thereof and extending proximally into the driver tip, and wherein the hexalobe engagement feature includes an alignment nub protruding distally beyond the distal end of the engagement feature; and
a hexalobe screw, the hexalobe screw including a head portion forming a proximal portion of the screw and a threaded portion forming the distal portion of the screw, wherein a socket is defined inward from a proximal end into the head portion, wherein the socket is sized and shaped to receive and engage the hexalobe engagement feature of the screw driver tip.
